(19) United States
(12) Patent Application Publication
(10) Pub. No.: US 2008/0097390 A1
(43) Pub. Date: Apr. 24, 2008

(54) Spring actuated delivery system

(75) Inventors: Bruno Dacquay, Irvine, CA (US); James Foster, Santa Ana, CA (US); Casey Lind, Irvine, CA (US); Cesario Dos Santos, Aliso Viejo, CA (US)

Correspondence Address: ALCON, IP LEGAL, TB4-8, 6201 SOUTH FREEWAY, FORT WORTH, TX, 76134, US

(73) Assignee: Alcon Manufacturing, Ltd.

(21) Appl. No.: 11/527,858

(22) Filed: Sep. 27, 2006

(51) Int. Cl. A61M 31/00; A61M 5/20

(52) U.S. Cl. 604/521; 604/135

(57) ABSTRACT

A disposable drug delivery device has a dispensing needle, a plunger, a spring, a shaft, and a lock mechanism. The plunger is engaged with the inner surface of the dispensing needle, is capable of sliding in the cavity of the dispensing needle, and is fluidly sealed to the inner surface of the dispensing needle. The spring is connected to the plunger via a shaft. The lock mechanism holds the shaft in a first position such that the spring is in a compressed state. When the lock mechanism releases the shaft, the spring extends providing a force that moves the shaft and connected plunger in a direction toward the tapered end of the dispensing needle thereby expelling the drug from the dispensing needle into an eye. The spring also provides a retaining force so that the plunger is retained in an extended position against the tapered end of the dispensing needle.

### FIELD OF THE INVENTION

The present invention relates to a device for injecting a drug into an eye and more particularly to a disposable, spring-actuated, ophthalmic drug delivery device.

### BACKGROUND OF THE INVENTION

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically manually made using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to puncture the eye tissue with the needle, hold the syringe steady, and actuate the syringe plunger (with or without the help of a nurse) to inject the fluid into the eye. The volume injected is typically not controlled in an accurate manner because the vernier on the syringe is not precise relative to the small injection volume. Fluid flow rates are uncontrolled. Reading the vernier is also subject to parallax error. Tissue damage may occur due to an “unsteady” injection. In addition, when the needle is removed from the eye, the drug may be drawn out of the wound if the plunger is retracted. Such reflux leads to imprecise dosing.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. With this type of dispenser, the volumes delivered are highly dependent on fluid viscosity, surface tension, and the specific dispensing tip. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. While precise, this dispenser is expensive and requires an electrical signal to be delivered to the dispensing mechanism.

U.S. Pat. No. 6,290,690 discloses a surgical system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

Despite these efforts, a need remains for a dependable, low cost system for injecting precise volumes of substances into the eye without reflux.

### SUMMARY OF THE INVENTION

In one embodiment consistent with the principles of the present invention, the present invention is a spring actuated delivery system having a dispensing member, a plunger, a spring, a shaft, and a lock mechanism. The dispensing member has an inner surface, an outer surface, and a tapered end. The inner surface defines a cavity for receiving a quantity of a substance. The dispensing member has a dispensing tip and a cannula. The plunger is engaged with the inner surface of the dispensing member and is capable of sliding in the cavity of the dispensing member. The plunger is fluidly sealed to the inner surface of the dispensing member. The spring is connected to the plunger via the shaft. The lock mechanism engages the shaft, holding it in a first position such that the spring is in a compressed state. When the lock mechanism is released, the spring extends thereby driving the shaft and connected plunger in a direction toward the tapered end of the dispensing member. The plunger expels the substance from the dispensing member into the eye. The plunger engages the tapered end of the dispensing member.

In another embodiment consistent with the principles of the present invention, the present invention is a disposable drug delivery device having a dispensing needle, a plunger, a spring, a shaft, and a lock mechanism. The dispensing needle has an inner surface, an outer surface, and a tapered end. The inner surface defines a cavity for receiving a quantity of a drug. The plunger is engaged with the inner surface of the dispensing needle, is capable of sliding in the cavity of the dispensing needle, and is fluidly sealed to the inner surface of the dispensing needle. The spring is connected to the plunger via a shaft. The lock mechanism holds the shaft in a first position such that the spring is in a compressed state. When the lock mechanism releases the shaft, the spring extends providing a force that moves the shaft and connected plunger in a direction toward the tapered end of the dispensing needle thereby expelling the drug from the dispensing needle into an eye. The spring also provides a retaining force so that the plunger is retained in an extended position engaging the tapered end of the dispensing needle.

In another embodiment consistent with the principles of the present invention, the present invention is a method for delivering a substance into an eye. The substance to be injected into the eye is heated by heating a dispensing member in contact with the substance. After the substance is heated to an appropriate temperature, a lock mechanism release is enabled. A shaft held by the lock mechanism is released thereby allowing a compressed spring to extend and push a plunger located in the dispensing member to expel the substance from the dispensing member. The plunger is retained in an extended position by the application of a spring force.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a prior art syringe.

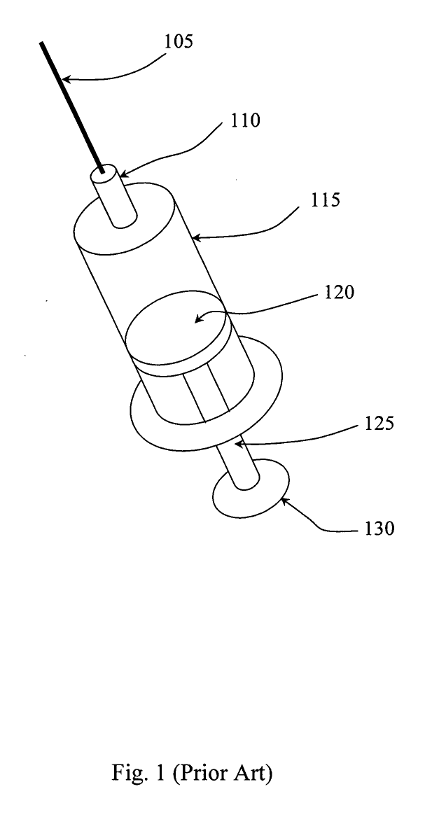

FIG. 2 is a cross section view of a spring actuated delivery system according to an embodiment of the present invention.

FIG. 3 is a cross section view of a spring actuated delivery system according to an embodiment of the present invention.

FIG. 4 is an exploded cross section view of a portion of spring actuated delivery system according to an embodiment of the present invention.

FIG. 5 is an exploded cross section view of a portion of a spring actuated delivery system according to an embodiment of the present invention.

FIG. 6 is a flow chart of one method of operation according to an embodiment of the present invention.

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

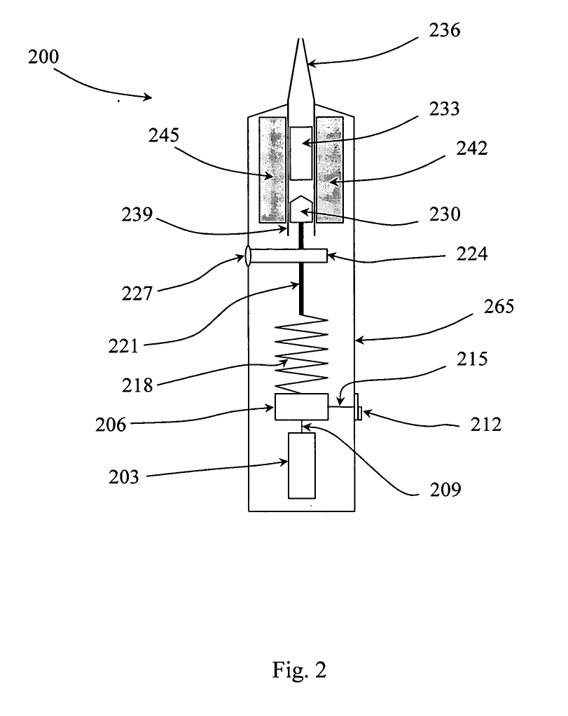

FIG. 2 is a cross section view of a spring actuated delivery system according to an embodiment of the present invention. In FIG. 2, the system 200 includes a spring 218, shaft 221, lock mechanism 224, lock mechanism release 227, plunger 230, substance 233, dispensing tip 236, and cannula 239. The system 200 may also optionally include battery 203, controller 206, switch 215, interfaces 209, 215, and heating mechanisms 242, 245.

In the embodiment depicted in FIG. 2, battery 203 is connected to controller 206 via interface 209. Switch 212 is connected to controller 206 via interface 215. One end of spring 218 is connected to shaft 221. The other end of spring 218 is affixed to a point on the interior of housing 265. This point may be located on an interior surface of housing 265, on a protrusion or connection site on the interior of housing 265, on controller 206, or in any other convenient location.

One end of shaft 221 is connected to spring 221. The other end of shaft 221 is connected to plunger 230. Shaft 221 is also engaged by lock mechanism 224. Lock mechanism 224 has a lock mechanism release 227. Lock mechanism release 227 is in the form of a button or switch located on housing 265. Plunger 230 is located in cannula 239. Plunger 230 is fluidly sealed to an interior surface of cannula 239. A substance to be injected into an eye is also located in cannula 239. Dispensing tip 236 is located on the distal end of cannula 239. Dispensing tip 236 and cannula 239 are one continuous structure for conveying substance 233. Dispensing tip 236 has an opening to allow substance 233 to be delivered into an eye. Collectively dispensing tip 236 and cannula 239 form a dispensing member.

Optional heating mechanism 242, 245 is located on either side of cannula 242. Alternatively, heating mechanism 242, 245 may surround cannula 239. Housing 265 encloses the parts as shown in FIG. 2.

In operation, the spring actuated delivery device 200 of FIG. 2 delivers substance 233 into an eye. Switch 212 is activated sending a signal via interface 215 to controller 206. Controller 206 controls the operation of heating mechanism 242, 245. When switch 212 is turned on, controller 206 turns on heating mechanism 242, 245. Controller 206 regulates the temperature of heating mechanism 242, 245 to properly heat substance 233. When heating mechanism 242, 245 is turned on, heat is transferred through cannula 239 to substance 233. Battery 203 provides power for the controller 206 and the heating mechanism 242, 245.

Controller 206 may also control whether lock mechanism release 227 can be activated. In this embodiment, controller 227 activates heat mechanism 242, 245 to heat substance 233 to a predetermined temperature. This predetermined temperature is a desired temperature for administration of substance 233 into an eye. For example, substance 233 may be in a solid, semi-solid, or viscous state at room temperature. Heat can be applied to substance 233 to decrease its viscosity so that it can be delivered through dispensing tip 236. Additionally, heat may be applied to substance 233 to raise it to a temperature at which it may be more effective when delivered into the eye.

In one embodiment, controller 206 heats substance 233 for a predetermined period of time. Since substance 233 is pre-loaded into spring actuated delivery system 200, the quantity of substance 233 is known as are the heat transfer characteristics of cannula 239. Therefore, heat from heat mechanism 242, 245 can be applied for a predetermined period of time to bring substance 233 up to the desired temperature.

In another embodiment, controller 206 receives an input that corresponds to the temperature of substance 233. In this configuration, a thermocouple, temperature senor, or other similar device measures the temperature of substance 233 or cannula 239. The measurement is sent to the controller 206.

Heating mechanism 242, 245 is optional. In some cases, it is not necessary to heat substance 233. In such a case, none of the heating steps occurs. In addition, the controller 206 may be absent in such a configuration as it is not necessary to have a controller to operate the heating mechanism 242, 245.

Dispensing tip 236 is inserted into the eye, typically through the pars plana region. Dispensing tip 236 is usually a needle, such as those used for drug delivery. Dispensing tip 236 typically has a trocar on its end to assist in the insertion of dispensing tip 236 into the eye.

After dispensing tip 236 is inserted into the eye, lock mechanism release 227 is activated. Lock mechanism release 227 may be in the form of a button or switch. When lock mechanism release 227 is activated, lock mechanism 224 releases shaft 221.

Before lock mechanism release 227 is activated, lock mechanism 224 holds shaft 221 in a first position such that spring 218 is in a compressed state. Since shaft 221 is connected to spring 218, in this position, the spring is ready to expand when lock mechanism 224 releases shaft 221.

When lock mechanism 224 releases shaft 221, spring 218 expands applying a force on shaft 221. Shaft 221 travels in a direction toward dispensing tip 236. Since shaft 221 is connected to plunger 230, the spring force provided by spring 218 pushes plunger toward dispensing tip 236. Plunger 230 contacts substance 233 and pushes it out of dispensing tip 236.

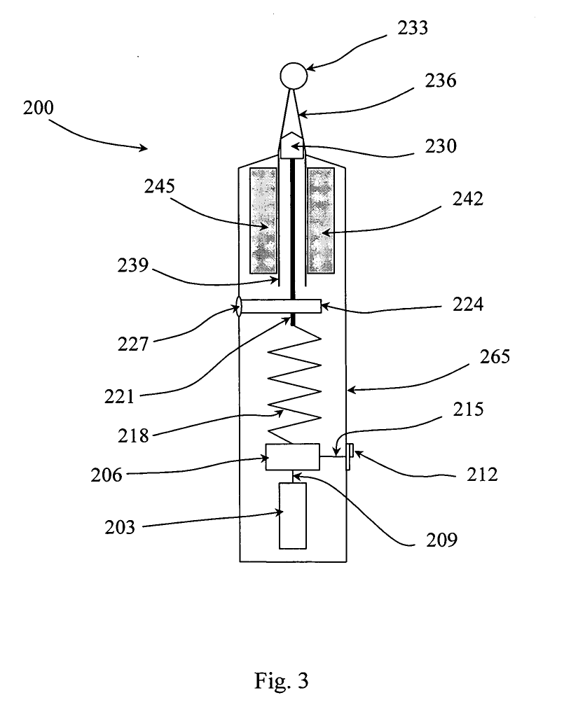

This is more clearly shown in FIG. 3. In FIG. 3, lock release mechanism 227 has been activated, lock mechanism 224 has released shaft 221, and spring 218 has expanded. The spring force provided by spring 218 has moved shaft 221 in a direction toward dispensing tip 236. Since plunger 230 is connected to shaft 221, plunger 230 was also moved in a direction toward dispensing tip 236. Substance 233 has been expelled from dispensing tip 236.

In FIG. 3, the taper in dispensing tip 236 prevents plunger 230 from continuing to travel toward dispensing tip 236. In this manner, the taper in dispensing tip 236 acts as a stop to prevent plunger 230 from exiting dispensing tip 236. In this position, spring 218 is less compressed than when it was in its first compressed position. However, even in this second extended position, spring 218 applies a force on shaft 221 that tends to keep plunger 230 is the position shown. In this manner, when dispensing tip 236 is removed from the eye, the plunger 230 does not move. The spring 218 holds the plunger 230 in an extended position. When the dispensing tip 236 is retracted from the eye, reflux can be avoided because the plunger is held in the extended position. It should also be noted that, depending on the shape of dispensing tip 236, some of substance 233 may remain in dispensing member 236.

Other types of stops for plunger 230 may also be employed. In other embodiments consistent with the principles of the present invention, a groove or ridge may be present on the interior surface of dispensing tip 236 or cannula 239. A corresponding ridge or groove may be present on plunger 230 or shaft 221. Alternatively, lock mechanism 224 may be re-engaged to hold shaft 221 and plunger 230 in a particular position.

The use of spring 218 assists in delivering the substance 233 into the eye in a smooth and steady manner. The flow rate of the substance 233 can be controlled by selecting a spring 218 with an appropriate spring constant. A faster flow rate can be achieved by selecting a spring with a larger spring constant. A slower flow rate can be achieved by selecting a spring with a smaller spring constant. The spring constant also depends on the amount of substance to be expelled from the dispensing tip, the viscosity of the substance, and the size of the dispensing tip and cannula.

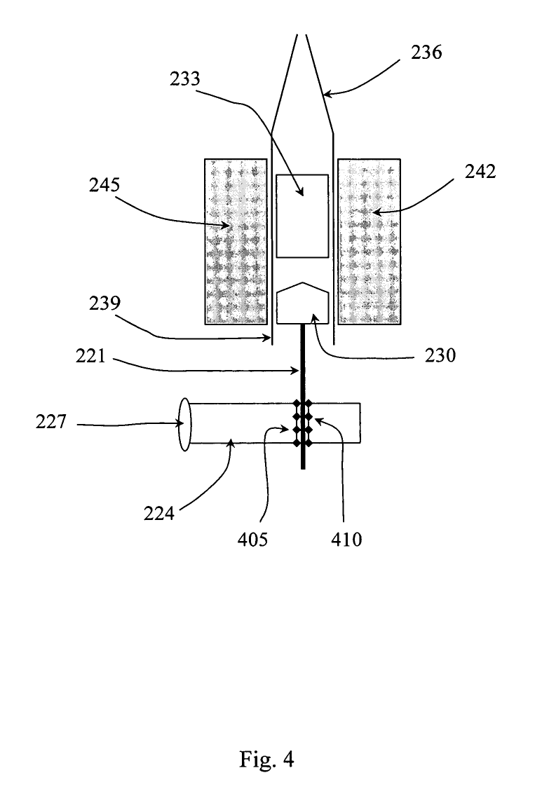

FIG. 4 is an exploded cross section view of a portion of a spring actuated delivery system according to an embodiment of the present invention. FIG. 4 depicts the dispensing tip end of the system and more particularly shows one embodiment of the lock mechanism 224. In FIG. 4, dispensing tip 236 and cannula 239 contain substance 233. Typically, substance 233 is a drug for treating a condition of the eye. Dispensing tip 236 and cannula 239 together form a needle suitable for injecting a drug into the eye. In one embodiment, the needle is a 25 gauge needle. The taper of dispensing tip 236 is such that it will engage plunger 230 when plunger 230 travels in a direction toward dispensing tip 236.

Plunger 230 rests in cannula 239 adjacent to substance 233. Plunger 230 is typically made of an elastomeric material so that it can be fluidly sealed to an interior surface of cannula 239. Shaft 221 is connected to plunger 230. Shaft 221 is typically made of a rigid polymer but may also be made of any other rigid material. In addition shaft 221 may have one or more linkages (not shown).

Lock mechanism 224 has two grips 405, 410 that hold shaft 221 in place. These two grips 405, 410 can be made of any suitable material that provides the necessary gripping capacity to hold shaft 221 in place. When actuated, lock mechanism release 227 releases grips 405, 410 and allows shaft 221 to be moved by the spring force.

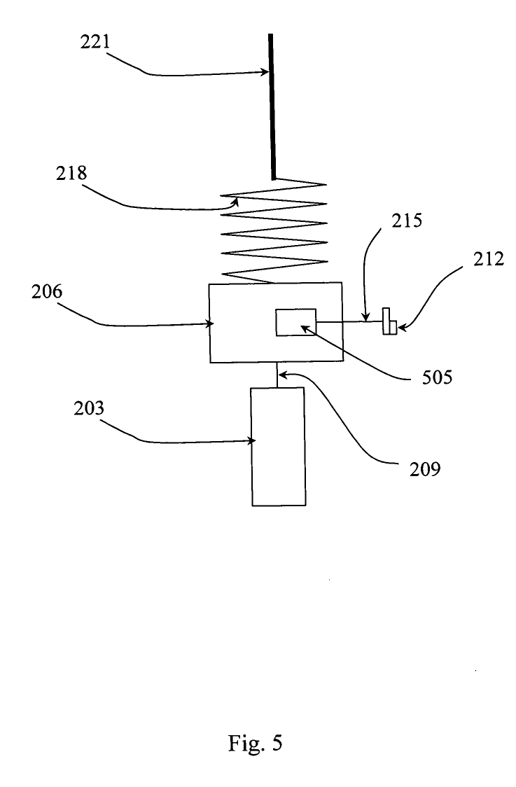

FIG. 5 is an exploded cross section view of a portion of a spring actuated delivery system according to an embodiment of the present invention. FIG. 5 depicts the controller end of the system. In FIG. 5, shaft 221 is connected to spring 218. Battery 203 is connected and provides power to controller 206. Switch 212 provides an input to controller 206 via interface 215.

Controller 206 includes a logic circuit 505. Logic circuit 505 receives an input from switch 212 via interface 215. Logic circuit 505 controls the operation of the heating mechanism. Typically, logic circuit 505 is a simple integrated circuit.

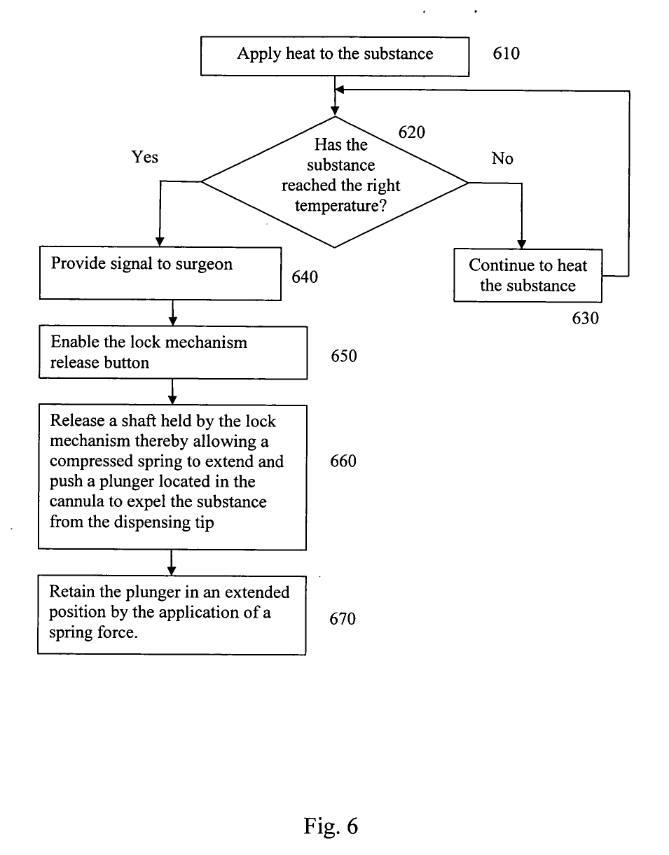

FIG. 6 is a flow chart of one method of operation according to an embodiment of the present invention. In 610, heat is applied to the substance. In 620, if the substance has not reached the proper temperature, then in 630, heat continues to be applied to the substance. If the substance has reached the proper temperature in 620, then in 640, a signal is provided to the surgeon or medical professional. This signal is typically in the form of an illuminated light emitting diode (“LED”) but may also be any type of visible or audible signal. In one embodiment, an LED is illuminated when the substance reaches the proper temperature.

In 650, the lock mechanism release is enabled. As previously discussed, the heating mechanism may run for a preset period of time before the lock mechanism is enabled or the system may have some form-of temperature sensing capabilities. Once the lock mechanism is enabled, the medical professional or surgeon can actuate the lock release mechanism to release the shaft.

In 660, the shaft held in place by the lock mechanism is released thereby allowing the compressed spring to extend and push the plunger located in the cannula. As the plunger moves, it expels the substance from the dispensing tip.

In 670, the plunger is retained in an extended position by the application of a force supplied by the spring. In this position, the plunger is engaged with the tapered end of the dispensing member.

The spring loaded delivery system 200 is appropriate as a single use consumable product. Such a disposable product can be assembled at a factory with a dosage of a substance installed. In this manner, the system allows for precise dosing. A precise volume of a substance can be preloaded into the delivery device. This helps to prevent dosing error on the part of the medical professional.

Additionally, proper storage and handling of the substance can be more easily assured. Since the substance is loaded into the system at the factory, the substance can be stored under precise conditions. Shipment of a preloaded system can also be under precise conditions.

From the above, it may be appreciated that the present invention provides an improved system and methods for delivering precise volumes of a substance into an eye. The present invention provides a single use, disposable delivery device that is capable of delivering a precise dosage without reflux. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A spring actuated delivery system comprising:
a dispensing member having an inner surface and an outer surface, the inner surface defining a cavity for receiving a quantity of a substance, the dispensing member having a tapered end;
a plunger engaged with the inner surface of the dispensing member, the plunger capable of sliding in the cavity of the dispensing member, the plunger fluidly sealed to the inner surface of the dispensing member;
a spring connected to the plunger via a shaft; and
a lock mechanism engaging the shaft, the lock mechanism holding the shaft in a first position such that the spring is in a compressed state;
wherein when the lock mechanism is released, the spring extends thereby driving the shaft and connected plunger in a direction toward the tapered end of the dispensing member and expelling the substance from the dispensing member into an eye, and further the plunger engages the tapered end of the dispensing member.

2. The system of claim 1 further comprising a release switch to release the lock mechanism.

3. The system of claim 2 further comprising a heating mechanism in thermal contact with the dispensing member.

4. The system of claim 3 further comprising control logic for controlling the operation of the heating mechanism wherein the control logic enables the release switch after the substance has reached an appropriate temperature.

5. The system of claim 4 further comprising a light emitting diode that is illuminated when the substance reaches the appropriate temperature.

6. The system of claim 1 wherein the substance is a drug for treating a condition of the eye.

7. The system of claim 1 wherein the dispensing member is a needle.

8. The system of claim 1 wherein the dispensing member is a cannula.

9. The system of claim 1 further comprising a battery.

10. A disposable drug delivery device comprising:
a dispensing needle having an inner surface and an outer surface, the inner surface defining a cavity for receiving a quantity of a drug, the dispensing member having a tapered end;
a plunger engaged with the inner surface of the dispensing needle, the plunger capable of sliding in the cavity of the dispensing needle, the plunger fluidly sealed to the inner surface of the dispensing needle;
a spring connected to the plunger via a shaft; and
a lock mechanism holding the shaft in a first position such that the spring is in a compressed state;
wherein when the lock mechanism releases the shaft, the spring extends providing a force that moves the shaft and connected plunger in a direction toward the tapered end of the dispensing needle thereby expelling the drug from the dispensing needle into an eye; and
further wherein the spring provides a retaining force so that the plunger is retained in an extended position engaging the tapered end of the dispensing needle.

11. The system of claim 10 further comprising a release switch to release the lock mechanism.

12. The system of claim 11 further comprising a heating mechanism in thermal contact with the dispensing member.

13. The system of claim 12 further comprising control logic for controlling the operation of the heating mechanism wherein the control logic enables the release switch after the substance has reached an appropriate temperature.

14. The system of claim 13 further comprising a light emitting diode that is illuminated when the substance reaches the appropriate temperature.

15. The system of claim 10 further comprising a battery.

16. A method for delivering a substance into an eye comprising:
heating the substance to be injected into the eye by heating a dispensing member in contact with the substance;
after the substance is heated to an appropriate temperature, enabling a lock mechanism release;
releasing a shaft held by the lock mechanism thereby allowing a compressed spring to extend and push a plunger located in the dispensing member to expel the substance from the dispensing member; and
retaining the plunger in an extended position by the application of a spring force.

17. The method of claim 16 further comprising:
providing a signal when the substance is heated to the appropriate temperature.

18. The method of claim 16 wherein the substance expelled is a drug for treating a condition of the eye.
